(19) United States
(12) Patent Application Publication
(10) Pub. No.: US 2010/0087709 A1
(43) Pub. Date: Apr. 8, 2010

(54) BALLOON CATHETER SHEATH

(76) Inventors: Arthur A. Bertolero, Danville, CA (US); Tamer Ibrahim, Pleasant Hill, CA (US)

Correspondence Address: GREGORY SMITH & ASSOCIATES, 3900 NEWPARK MALL ROAD, 3RD FLOOR, NEWARK, CA, 94560, US

(21) Appl. No.: 12/630,740

(22) Filed: Dec. 3, 2009

Related U.S. Application Data
(62) Division of application No. 10/988,025, filed on Nov. 12, 2004, now abandoned.
(60) Provisional application No. 60/519,766, filed on Nov. 12, 2003.

(51) Int. Cl. A61B 1/00

(52) U.S. Cl. 600/116

(57) ABSTRACT

Apparatus and method for using disposable jackets or sheaths with many kinds of surgical devices, such as catheters and endoscopes. An inflatable balloon is mounted on an outside surface of the sheath. The sheath may also include radiopaque markers, a sterility strip, one or more attachment clips, adhesive attachment, an endcap, and/or an inflation lumen formed as a tube that extends beyond the proximal end of the sheath. The balloons of the sheath may have regular or irregular profiles and shapes, including but not limited to: a bowtie shape, a donut shape, helix, spiral, alternating on opposing sides. The balloon may be simultaneously inflated through a common lumen or they may separately inflatable through separate lumens.

### CROSS REFERENCE TO OTHER APPLICATIONS

This application is a divisional of U.S. application Ser. No. 10/988,025, filed Nov. 12, 2004, which claims the benefit of U.S. provisional patent application Ser. No. 60/519,766, filed Nov. 12, 2003, which are hereby incorporated by reference in their entirety.

### FIELD OF THE INVENTION

This invention is directed towards jackets or sheaths for use on catheters to add functionality to the catheters on which they are used. More particularly, it is directed toward a sheath having one or more balloon located on a distal end thereof and methods of use.

### BACKGROUND OF THE INVENTION

Catheters are used for a variety of surgical procedures. Procedures using catheters are performed for a variety of purposes, including diagnostic, interventional, and other therapeutic procedures. During many of these procedures, it is necessary to keep the distal end of the catheter in a relatively stable position to perform the desired procedure. One solution to ensure that a catheter is maintained in the proper position is to use an expandable balloon disposed near the distal end of the catheter shaft.

Balloon catheters typically include a lumen that extends from a proximal end to the balloon end and provides fluid to the balloon for its inflation. Once the procedure is complete, the fluid is removed from the balloon, thereby deflating the balloon and allowing the catheter to be removed. Although various types of balloon anchored catheters have been proposed, they often suffer from one or more limitations. What is needed is a jacket or sheath that can deploy balloon structures to improve stabilization of the catheter and that may be useable to add other functionality to the catheter.

### SUMMARY OF THE INVENTION

The present invention includes disposable jackets or sheaths for use with many kinds of surgical devices, but is particularly beneficial for use on catheters, endoscopes, and the like (hereafter the term catheter will be used to include all such devices) to add functionality to the devices on which they are used. The medical devices and the sheaths may be disposable or reusable. The distal end of the sheath may be open or closed depending on the medical procedure being performed. If the sheath is used over a viewing scope and the procedure to be performed is viewing, the end of the sheath may be closed or capped.

In one embodiment, the sheath includes: an elongated body having a proximal end and a distal end, a main lumen extending through the elongated body from the proximal end to the distal end, the main lumen is sized and configured to allow insertion of a medical instrument therethrough, and an inflatable balloon is mounted on an outside surface of said elongated body proximate the distal end of the sheath.

A second embodiment is a sheath having an elongated body having a proximal end and a distal end, a main lumen extending through the elongated body from the proximal end to the distal end, the main lumen is sized and configured to allow insertion of a medical instrument therethrough, an inflatable balloon mounted on an outside surface of the elongated body proximate the distal end, an inflation lumen extending from the proximal end to the inflatable balloon, a bendable region of the elongated body, the bendable region being located intermediate the proximal end and the distal end, and a frangible region forming a portion of said elongated body. The frangible region of the sheath may be a circumferential line around said elongated body or along a longitudinal axis of said elongated body.

The sheath may be used in combination with endoscopes or catheters. The main lumen of the sheath is sized and configured to conform to the exterior of the medical instrument being used. The sheath may include one or more of the following additional features: radiopaque markers located within the wall, a sterility strip located on an exterior surface, one or more clips sized and configured to hold said sheath to a medical instrument, an inflation lumen formed as a tube that extends beyond the proximal end of the sheath.

The balloons of the sheath may have regular or irregular profiles and shapes, including but not limited to: a bowtie shape, a donut shape, helix, spiral, or may alternate on opposing sides of the sheath. The balloons may be simultaneously inflated through a common lumen or they may separately inflatable through separate lumens.

A method of using one of the balloon sheath described herein includes the steps of inserting a medical instrument into a main lumen of the balloon sheath; inserting the medical instrument and the balloon sheath into a patient; inflating the balloon located proximate a distal end of the sheath; and performing a surgical procedure with the medical instrument. If needed, the end of the medical instrument may be cleaned by pumping irrigation fluid through the irrigation lumen. The irrigation solution may then be withdrawn through the suction lumen by a suction or vacuum source.

### BRIEF DESCRIPTION OF THE DRAWINGS

FIG. 1A shows an example sheath fitted over a generic endoscope.

FIG. 1B shows the balloons of FIG. 1A sucked down onto the sheath.

FIG. 1C shows the balloons of FIG. 1A inflated.

FIG. 2A shows another balloon sheath embodiment fitted over a catheter of scope.

FIG. 2B shows the balloon sheath embodiment of FIG. 2A in isolation.

FIG. 3A shows an embodiment with separately inflatable balloon chambers.

FIG. 3B shows an alternate embodiment of FIG. 3.

FIG. 4A shows an example balloon configuration.

FIG. 4B shows an alternate example balloon configuration.

FIG. 5A shows another embodiment of the sheath of the invention.

FIG. 5B is a cross section of the sheath of FIG. 5A.

FIG. 6A shows an embodiment of the sheath having an expandable channel.

FIG. 6B shows the sheath of FIG. 6A in the open position.

FIG. 7 shows another embodiment of the invention.

### DETAILED DESCRIPTION

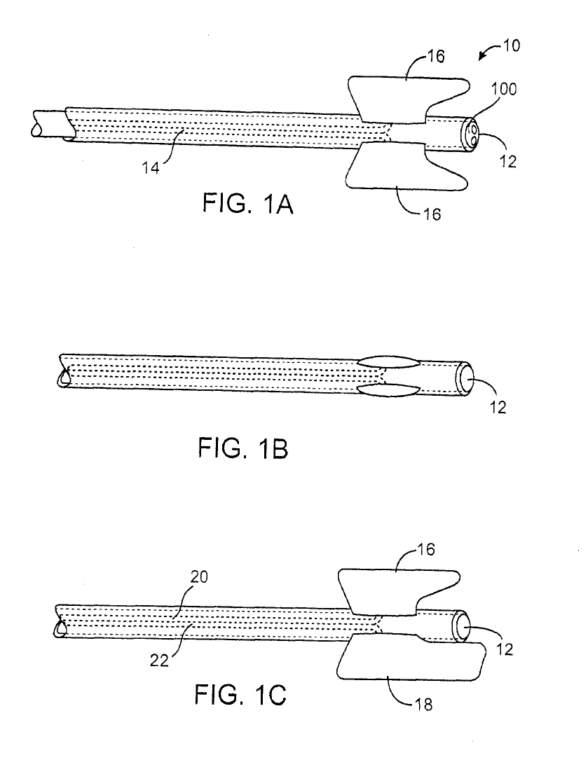

The sheaths of the present invention include disposable jackets or sheaths for use with many kinds of surgical devices, but is particularly beneficial for use on catheters, endoscopes, and the like (hereafter the term catheter will be used to include all such devices) to add protection and/or functionality to the devices on which they are used. The sheaths may be disposable or reusable. FIG. 1A shows an example sheath 10 fitted over a generic endoscope 100. The endoscope 100 is inserted into the main channel or lumen 12 of the sheath 10. In this embodiment, the sheath 10 includes lumens 14 for inflating the balloons 16. The balloons 16, 18 may be connected to separate lumens 20, 22, as seen in FIG. 1C, thereby allowing separate and/or different inflation of the two balloons 16, 18. Alternately, the lumen may branch to reach multiple balloons 16. In alternate embodiments, the balloons 16 may be coupled to the catheter or scope 100 and be inflated by a lumen within the catheter 100; however, in the version shown, one or more lumens 14, 20, 22 in the sheath 10 are used to inflate the balloons 16, 18. FIG. 1B shows the balloons 16 of FIG. 1A sucked down onto the sheath 10 for insertion. FIG. 1C shows the balloons 16, 18 inflated. In this embodiment, the separate balloons 16, 18 may be inflated to different sizes, as seen in FIG. 1C. Additional inflation could be used to create a widening opening or extend the length of the opening depending on the configuration of the balloons.

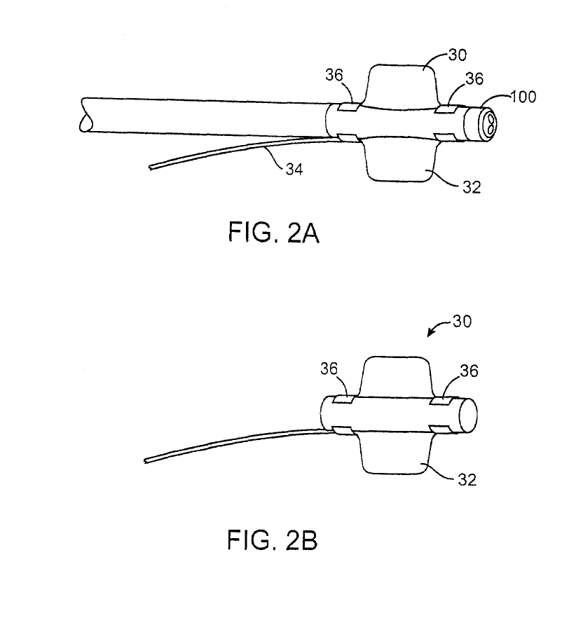

FIG. 2A shows another balloon sheath 30 embodiment fitted over a catheter or scope 100. In this embodiment, the sheath 30 is shorter. The balloon 32 covers the majority of the surface area of the sheath 30, and a separate lumen 34 is coupled to the balloon 32. Optionally, clips 36 at either end of the sheath 30 may be used to attach the sheath 30 to the catheter 100. FIG. 2B shows the balloon sheath 30 embodiment of FIG. 2A in isolation.

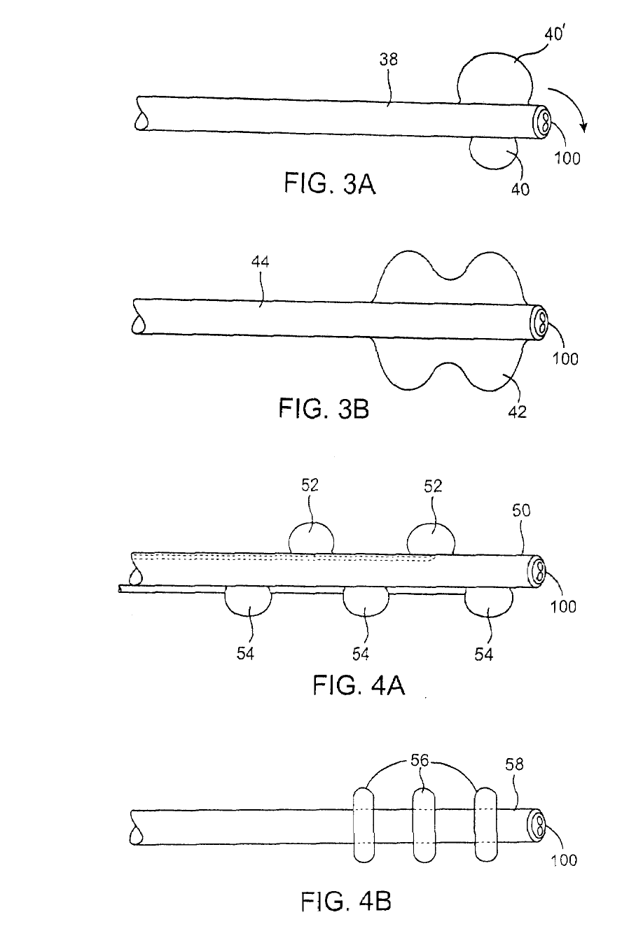

The balloons of the invention may have more than one compartment, which may be inflated individually, or all compartments may be inflated at once. For example, FIG. 3A shows an embodiment of the sheath 38 with separately inflatable balloon chambers 40, 40′. Differential inflation of the balloons 40, 40′ may provide advantages including but not limited to assisting in steering the catheter 100.

The balloons may have a variety of shape configurations when inflated depending on the planned function of the balloon. For example, the balloons may have a consistent diameter, or one or more reduced diameter sections creating a “bow tie” profile for the balloon 42, as seen in FIG. 3B, which may be especially helpful to enable positioning the balloon 42 on both sides of an anatomic structure, thereby potentially assisting the sheath 44 in gripping an anatomic structure during use.

FIGS. 4A and 4B show two example configurations having multiple balloons. In FIG. 4A, multiple balloons are arranged along the length of the sheath 50. The balloons 52, 54 are alternately located on opposite sides of the sheath 50. This configuration could be used in situations where the balloons 52, 54 are used to steer the sheath 50. The balloons 52 on one side could be inflated to push the tip of the sheath 50 in one direction and then the other set of balloons 54 could be inflated to push the tip of the sheath 50 in the opposite direction. This configuration could also be used to push the catheter 100 to one side of an anatomical opening to improve the line of site viewing or advance the catheter 100 and sheath 50.

In FIG. 4B, a series of toroid or donut shaped balloons 56 are located along the length of the sheath 58. The balloons 56 may be the same or different sizes along the length. For example, the balloons 56 could be of increasing size toward the distal end or toward the proximal end. This could be used in cases where the tissue opening is conical or a conical opening is desired. The balloons 56 may be located closely together to form a solid or almost solid, ridged surface, or the balloons 56 may be spaced apart to provide one or more large spaces between the balloons 56. If desired, the spaces may be particularly sized and spaced to rest on opposite sides of a specific anatomic structure.

The sheaths 50, 58 of FIGS. 4A and 4B may also be suitable configurations to provide additional traction. Each of the balloons 52, 54, 56 would press into the surrounding tissue thereby holding the sheath 50, 58 in place. Any irregularities in the tissue would be held between the multiple balloons 52, 54, 56, thereby stabilizing the sheath 50, 58 and the corresponding medical instrument 100 located within the sheath 50, 58.

The balloons may be arranged in many configurations on the sleeve, including but not limited to being arranged longitudinally as described above. Alternately, one or more balloons may be arranged to form a spiral or helical ridge. The balloons may be fixed or moveable. The balloons and inflation/deflation lumens may be formed integral with the sheath, or added in a second manufacturing procedure.

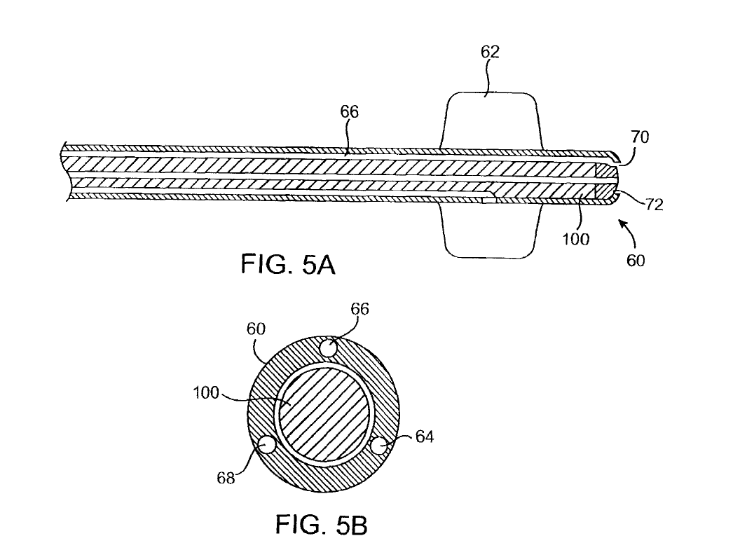

FIGS. 5A and 5B show an embodiment of the invention wherein the sheath 60 includes at least one balloon 62, an inflation lumen 64 and several optional lumens. The inflation lumen 64 may be used to inflate and deflate the balloon 62. An irrigation lumen 66 may be used to direct saline or another flushing agent toward to distal end of the catheter or scope 100 to irrigate the work area and/or to flush the lens of the catheter 100. The outlet 70 for the irrigation lumen 66 may be directed toward the end of the sheath 60 as shown, or the outlet 70 may be rotatable or steerable to allow the user to direct the irrigation solution toward a particular tissue structure or toward a part of the sheath 60 or instrument 100 within the sheath 60. A suction lumen 68 may be connected to a suction pump to withdraw the irrigation solution as it is used. A lens 72 may be placed over the end of the sheath 60 to protect the tip of a view scope 100.

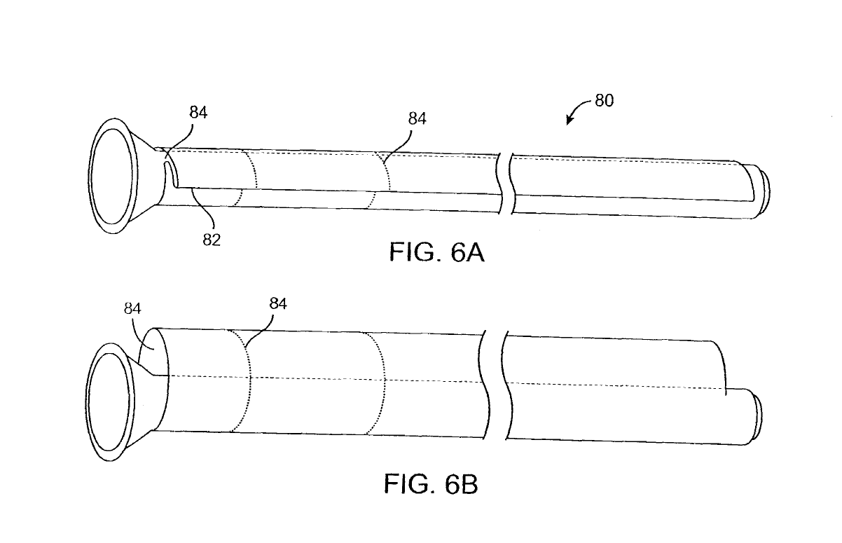

FIGS. 6A and 6B show an embodiment of the sheath 80 including a longitudinal fold 82 or lumen. The additional lumen may be used for an additional medical device, such as another catheter or scope. Alternately, the opening may be connected with the main channel 84, as shown, to allow an extra large scope to be used or to allow an additional instrument to be inserted adjacent the catheter or scope 100. The sheath 80 shown also includes frangible zones 84 that create break lines allowing the sheath 80 to be shortened to fit a particular scope or a particular procedure. The break lines 84 may be cut lines to direct where the sheath 80 may be cut or the break lines 84 may be an area weaken to an extent that the user may tear the necessary sections off of the main body of the sheath 80.

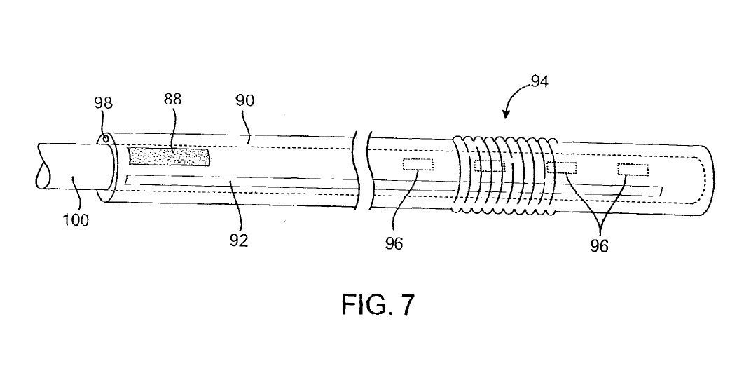

FIG. 7 shows an embodiment of the sheath 90 that includes several additional optional features. A sterilization strip 88 may be included on the sheath 90 so that upon visual inspection of the sheath 90, it can be determined if the sheath 90 has been sterilized. A frangible line or region 92 may be added along the length of the sheath 90 to aid in removal of the sheath 90 from the catheter 100 inserted therein. A bendable region 94 may be added. Although the bendable region 94 may use any standard technology to form to create the bendable region 94, the current embodiment shows a series of corrugations to create the bendable region 94. Radiopaque markers 96 may also be placed along the length of the sheath 90, at the distal end or at other specific points along the length of the sheath 90, such as at the bendable region 94 to aid in identifying the position and orientation of the sheath 90 and the catheter or scope 100 located within. An additional lumen 98 may be used to pass a small diameter medical instrument through the sheath 90 to the working region located at the distal end of the catheter.

An additional embodiment of the invention includes a repositionable balloon on the distal end of the sheath so that the catheter may be advanced without losing the originally created space. In some embodiments, the balloon that may be reshaped or reconfigured to develop certain space in the closed shape. The particular shape of the balloon may be reshaped as so to enhance exposure to the posterior side of the heart.

The balloons used in the invention may be made from a compliant, non-compliant, or combination of materials. Examples of compliant balloon materials include but are not limited to polyethylene; polyurethane; Tecoflex®; or any combination of these or other suitable materials. Examples of non-compliant balloon materials include nylon; polyester (PET polyethylene terephthalate, or other); Pebax®; polyimide; polyamide; or any combination of these or other suitable materials.

Depending on the instrument to be used, the sheath may be formed of a flexible material to conform to a flexible device, or a rigid, semi-rigid or malleable material, as desired. The sheath may be fabricated using any of a number of methods known to those skilled in the art, including but not limited to, dip coating, spraying, extrusion, molding, combination or other suitable process or method.

In addition to the embodiments of the sheath shown and described herein, any of the embodiments may also include the following features. The sheath may include one or more reduced thickness wall sections or perforations that can split apart when desired to enable easier removal of the sheath from around the catheter, endoscope or other instrument. The sheath may have a designated area to tear or cut to shorten the length of the sheath. The sheath may have at least one area that is designed to allow the endoscope to bend, without being constricted by the sheath, by having a more compliant section, and or a feature such as an accordion or bendable straw (corrugated) configuration section or sections. The sheath may have at least one lumen, channel, slot and/or other channel to allow the insertion of an instrument or device for diagnostic, therapeutic or other purposes. The distal tip of the sheath may be covered with one or more layers of the sheath material, and or a lens component. The sheath may contain radiopaque markers, combined with the sheath material, and or painted, printed or bonded onto the sheath. The sheath may include a sterilization indicator, to insure that a sterile sheath is being used. The sheath may contain at least one radial, circumferential perforation where the sheath length can be modified by tearing along the perforation and removing the unwanted length. The sheath may have at least one adhesive or bonding layer, on the inside diameter, and or outside diameter, that may have a release liner that can be removed, to tack or temporarily bond the sheath to the catheter. The sheath may be formed with at least one longitudinal fold, to allow catheters of different diameters to be used. For example, when using a large diameter endoscope, the folds will open as the catheter is inserted, accommodating the larger catheter size. The sheath (and or balloon on said sheath) may be coated with an antibacterial, lubricious, or other type of coating.

Methods for using the invention include a method of using a rigid endoscope to visualize the placement of a flexible endoscope or catheter; using the superior optics of a rigid endoscope in combination with the superior flexibility to visualize anatomical structures for a surgical procedure; using a rigid endoscope to visualize and create an opening to the heart and using a flexible scope to visualize the back of the heart; creating an opening or openings for the delivery of the scopes, where the scopes are placed through a single insertion point or through separate openings depending on the surgical procedure; a method of combining the two images in a picture-in-picture format to facilitate the surgical procedure.

The balloons maybe used for a variety of different purposes including, but not limited to occluding lumens; tissue resection; fluid (or gel) infusion (to clear the end of sheath/visualization instrument, or diagnostic, therapeutic or other desired fluid or gel); to create a void or space within the body to create a line-of-sight in front of a viewing instrument, or to move or separate tissues in order to provide clear line of site, or other visualization purposes; to provide opposite anatomic structure wall support; steering or guidance of the catheter or other device; protection of the distal end of the catheter; or a combination of these and/or other uses.

A method of using one of the balloon sheath described herein includes the steps of inserting a medical instrument into a main lumen of a balloon sheath; inserting the medical instrument and balloon sheath into a patient; inflating a balloon located proximate a distal end of the sheath; and performing a surgical procedure with the medical instrument. If needed, the end of the medical instrument may be cleaned by pumping irrigation fluid through the irrigation lumen. The irrigation solution may then be withdrawn through the suction lumen by a suction or vacuum source.

Many features have been listed with particular configurations, options, and embodiments. Any one or more of the features described may be added to or combined with any of the other embodiments or other standard devices to create alternate combinations and embodiments.

While the present invention has been described herein with respect to the exemplary embodiments and the best mode for practicing the invention, it will be apparent to one of ordinary skill in the art that many modifications, improvements and subcombinations of the various embodiments, adaptations and variations can be made to the invention without departing from the spirit and scope thereof. Thus, the examples given should only be interpreted as illustrations of some of the preferred embodiments of the invention, and the full scope of the invention should be determined by the appended claims and their legal equivalents.

What is claimed is:

1. A sheath for use with a medical device during a medical procedure, the sheath comprising:
an elongated body having a proximal end and a distal end,
a main lumen extending through said elongated body from said proximal end to said distal end, said main lumen sized and configured to allow insertion of a medical instrument therethrough,
and an inflatable balloon mounted on an outside surface of said elongated body proximate said distal end.

2. The sheath of claim 1, used in combination with said medical instrument, wherein said medical instrument is an endoscope, and wherein said main lumen is sized to allow said endoscope to fit therein.

3. The sheath of claim 2, further comprising an inflation lumen located within said endoscope, said inflation lumen having a distal end connectable with said inflatable balloon and a proximal end connectable with a source of inflation solution.

4. The sheath of claim 1, used in combination with said medical instrument, wherein said medical instrument is a catheter, and wherein said main lumen is sized to allow said catheter to fit therein.

5. The sheath of claim 1, further comprising an inflation lumen extending from said proximal end of said elongated body to said inflatable balloon.

6. The sheath of claim 1, further comprising an adhesive located within the main lumen.

7. The sheath of claim 1, further comprising an endcap covering the distal end of the main lumen.

8. The sheath of claim 1, further comprising a frangible region forming a portion of said elongated body.

9. The sheath of claim 8, wherein said frangible region is a circumferential line around said elongated body.

10. The sheath of claim 8, wherein said frangible region is along a longitudinal axis of said elongated body.

11. The sheath of claim 1, further comprising a clip sized and configured to hold said sheath to the medical instrument.

12. The sheath of claim 11, wherein a proximal end of said inflation lumen is formed by a tube extending beyond said proximal end of said elongated body.

13. The sheath of claim 1, wherein said balloon has an irregular profile.

14. The sheath of claim 13, wherein said profile has a depression forming the balloon into a bowtie shape.

15. The sheath of claim 1, further comprising a plurality of additional inflation balloons and wherein said inflation balloons are located along a length of said elongated body.

16. The sheath of claim 15, wherein said inflatable balloons are alternately located on opposite sides of said elongated body.

17. The sheath of claim 1, further comprising a plurality of additional inflation balloons and wherein said inflation balloons are donut shaped and located in spaced apart relation along a length of said elongated body.

18. The sheath of claim 1, further comprising a second inflatable balloon and wherein said inflatable balloons are independently inflatable.

19. The sheath of claim 1, further comprising a second inflatable balloon and wherein said inflatable balloons have different shapes.

20. The sheath of claim 1, wherein said main lumen is expandable.
